(19) United States
(12) Patent Application Publication
(10) Pub. No.: US 2026/0315274 A1
(43) Pub. Date: Oct. 15, 2026

(54) PTOSIS CLAMP

(71) Applicant: The Regents of the University of California, Oakland, CA (US)

(72) Inventors: Taras Gout, Oakland, CA (US); Robert Goldberg, Oakland, CA (US)

(21) Appl. No.: 19/485,508

(22) PCT Filed: May 30, 2024
(86) PCT No.: PCT/US2024/031547

§ 371 Date: Nov. 18, 2025

Related U.S. Application Data
(60) Provisional application No. 63/504,883, filed on May 30, 2023.

(51) Int. Cl. A61F 9/007; A61B 17/02; A61B 17/30

(52) U.S. Cl. CPC A61F 9/00718; A61B 17/0231; A61B 17/30

(57) ABSTRACT

A ptosis clamp device is described. The device includes a first lever having a proximal end and a distal end, a second lever having a proximal end and a distal end, with the first and second levers fixed at their proximal ends to form a fulcrum. The device further includes a clamping bar with a first junction connecting the distal end of the first lever to the first clamping bar, a second clamping bar with a second junction connecting the distal end of the second lever to the second clamping bar. The angle of the levers relative to the clamping bars is between 40-250°. In some embodiments, the device further includes a locking mechanism.

### CROSS-REFERENCE TO RELATED APPLICATIONS

This application claims priority to U.S. Provisional Application No. 63/504,883, filed on May 30, 2023, incorporated herein by reference in its entirety.

### BACKGROUND OF THE INVENTION

Ptosis is a medical condition wherein the upper eyelid lies abnormally low or droops, due in part to the elongation of the inner eyelid Müller's muscle. The condition can obstruct and blur the field of vision, depending on severity.

In many instances, a surgical procedure is performed on the eye tissue to tighten the muscle that lifts the upper eyelid. Depending on severity of the condition, a Müller's muscle conjunctival resection, or a shortening of the levator palpebrae or levator muscle advancement may be performed. Practitioners (e.g., surgeons) generally use conventional surgical forceps to grasp the excess Müller's muscle while suturing the tissue directly below the clamp and resecting the muscle in between the sutures and clamp.

Ptosis clamps have been around for decades (e.g., the Putterman clamp), with the overall shape and design going relatively unchanged. Unfortunately, these existing ptosis clamp designs have operational and ergonomic challenges due to a limited range of motion and positioning in the practitioner's hand. These design flaws result in poor visualization of the surgical site, as well as increasing unwanted trauma to the eye tissue due to clamping or simultaneous manipulation with additional instruments.

Thus, there is a need in the art for an improved ptosis clamp device that is safe, easy to operate, improves overall visualization and requires less tissue manipulation. The present invention satisfies this need.

### SUMMARY OF THE INVENTION

Aspects of the present invention relate to a ptosis clamp device having a first lever having a proximal end and a distal end, a second lever having a proximal end and a distal end, wherein the first and second levers are fixed at their proximal ends to form a fulcrum, a first clamping bar, a first junction connecting the distal end of the first lever to the first clamping bar, a second clamping bar, and a second junction connecting the distal end of the second lever to the second clamping bar, wherein a first angle of the first lever relative to the first clamping bar is between 40-250°, wherein a second angle of the second lever relative to the second clamping bar is between 40-250°, and wherein the first and second angles are equal.

In some embodiments, the first and second angles are between 50-200°. In some embodiments, the first and second angles are between 60-180°. In some embodiments, the first and second angles are between 90-150°. In some embodiments, the first and second angles are adjustable within the first and second junctions, respectively.

In some embodiments, the first and second clamping bars each have at least one curved portion. In some embodiments, the first and second junctions each have at least one curved portion.

In some embodiments, the first and second clamping bars each have an opposing, inner facing surface that is smooth. In some embodiments, the first and second clamping bars each have an opposing, inner facing surface that is textured. In some embodiments, each texture surface includes any of indents, grooves, ridges, detents, or serrations.

In some embodiments, the first and second clamping bars each have an opposing, inner facing surface with one or more pins extending outward from each surface. In some embodiments, the first and second bars each have one or more openings opposing each pin and at least partially passing through the respective bar. In some embodiments, each opening passes fully through the respective bar. In some embodiments, each opening has a chamfered portion.

In some embodiments, the ptosis device includes a locking mechanism attached to the first and second levers configured to releasably lock the levers together. In some embodiments, the locking mechanism includes a receiver attached to the first lever, an opening in the second lever, and a catch slidably attached to the second lever over the opening, wherein the receiver at least partially passes through the opening and engages the catch. In some embodiments, the opening in the second lever has a chamfered portion configured to align the receiver to the center of the opening. In some embodiments, the catch has a ramped portion having a ramp with a catch opening configured to releasably engage the receiver in one or more positions on the ramp. In some embodiments, the receiver has a distal flanged portion configured to engage with the catch opening and ramp. In some embodiments, the catch opening has sidewalls forming a proximal narrow portion and a distal wider portion.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIGS. 1A, 1B & 1C depict various views of an exemplary ptosis clamp device according to aspects of the present invention. FIG. 1A is an image depicting a right perspective view of an exemplary ptosis clamp device. FIG. 1B is an image depicting a left perspective view of an exemplary ptosis clamp device. FIG. 1C is an image depicting a right perspective view of an exemplary ptosis clamp device wherein the device is closed and locked.

FIGS. 2A, 2B & 2C depict various views of an exemplary junction region and clamping portion for a ptosis clamp device according to aspects of the present invention. FIG. 2A is an image depicting a side view of an exemplary junction region and clamping portion. FIG. 2B is an image depicting a side view of an exemplary junction region and clamping portion. FIG. 2C is an image depicting a right perspective view of an exemplary junction region and clamping portion.

FIGS. 3A, 3B & 3C depict various views of an exemplary ptosis clamp device according to aspects of the present invention. FIG. 3A is an image depicting a side view of an exemplary ptosis clamp device. FIG. 3B is an image depicting a side view of an exemplary ptosis clamp device. FIG. 3C is an image depicting a front view of an exemplary ptosis clamp device.

FIGS. 4A, 4B & 4C depict various views of an exemplary locking mechanism for a ptosis clamp device according to aspects of the present invention. FIG. 4A is an image depicting a right perspective view of an exemplary locking mechanism. FIG. 4B is an image depicting a left perspective view of an exemplary locking mechanism. FIG. 4C is an image depicting a right perspective view of an exemplary locking mechanism wherein the locking mechanism is closed and locked.

FIG. 5 depicts an exploded front perspective view of an exemplary ptosis clamp device according to aspects of the present invention.

FIGS. 6A & 6B are images of alternative exemplary ptosis clamp devices according to aspects of the present invention.

FIG. 7 is an image of an exemplary ptosis clamp device wherein the angle of the clamping portion relative to the lever arms or grasping handle is adjustable.

FIG. 8 is an image of a prototype of an exemplary ptosis clamp device.

FIG. 9A is an image of the prototype device of FIG. 8 with the clamping portion positioned at the patient's upper eyelid and the lever arms or grasping handle extending across the midline over the bridge of the patient's nose.

FIG. 9B is an image of the prototype device of FIG. 8 with the clamping portion engaging the underside of the patient's eyelid.

FIG. 9C is an image depicting the positioning of a Putterman clamp on the upper eyelid of the patient's left eye as compared to the positioning of the prototype device of FIG. 8 on the upper eyelid of the patient's right eye.

FIG. 9D is an image of the positioning of a practitioner's hands relative to the patient's head when using a Putterman clamp in conjunction with a second instrument.

FIG. 9E is an image of the positioning of a practitioner's hands relative to the patient's head when using the prototype device of FIG. 8 in conjunction with a second instrument. As shown, the prototype device of FIG. 8 can engage the patient's eyelid with the lever arms or grasping handle extending away from the midline of the patent's head, thereby permitting full visualization of the area of interest by the practitioner and greater access with the second instrument.

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements found in systems and methods of a ptosis clamp device. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The terms “patient,” “subject,” “individual,” and the like are used interchangeably herein, and refer to any animal amenable to device and methods described herein. The patient, subject or individual may be a mammal, and in some instances, a human.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, +5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As contemplated herein, the ptosis clamp device of the present invention improves the efficacy of various surgical procedures, including in one example, posterior ptosis surgery.

Referring now in detail to the drawings, in which like reference numerals indicate like parts or elements throughout the several views, in various embodiments, presented herein is a ptosis clamp device.

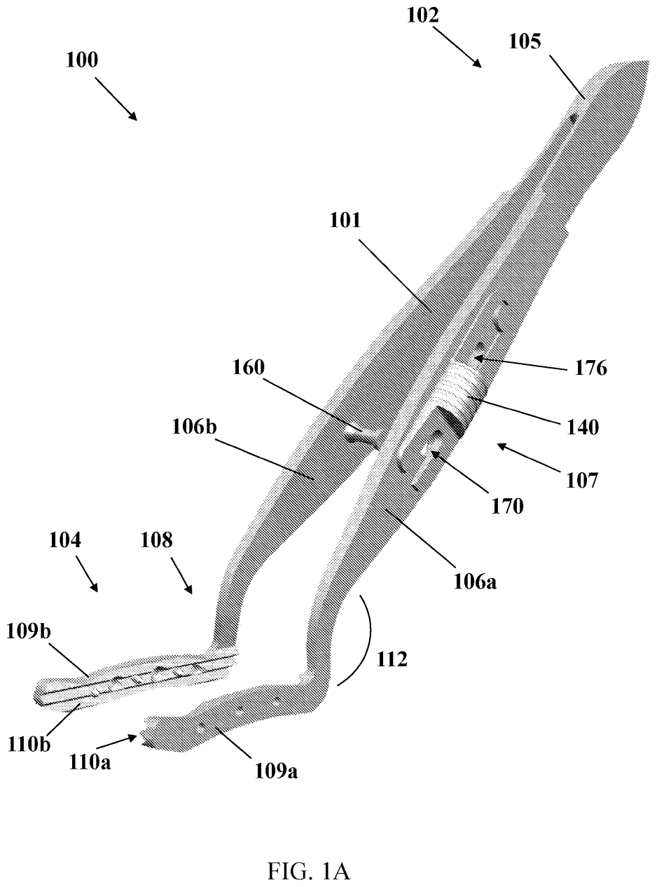

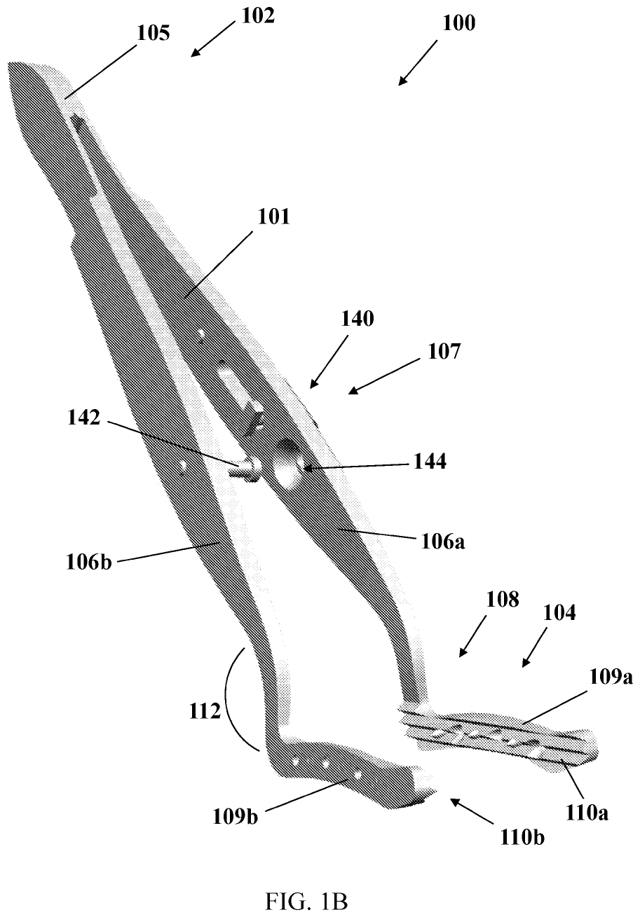

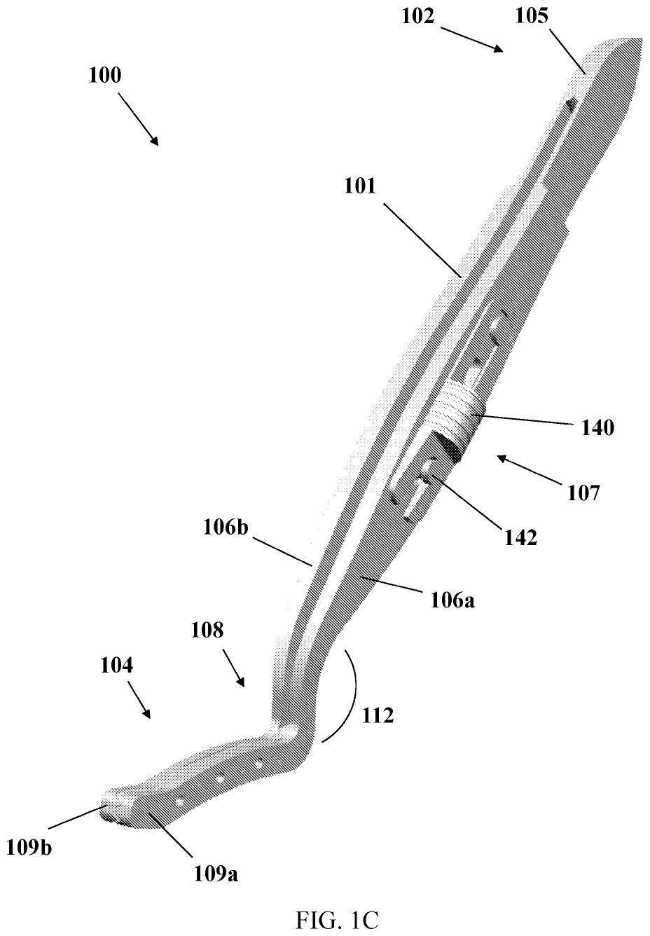

Referring now to FIGS. 1A through 1C, an exemplary embodiment of a ptosis clamp device 100 is presented. As contemplated herein, a ptosis clamp device 100 may be hand-driven forceps used by a practitioner or other practitioner to clamp onto, hold, push aside, or manipulate a patient's skin or other tissue during a procedure, such as ptosis repair. Ptosis clamp device 100 includes a body 101 which generally serves as a grasping handle or grasping region. Body 101 functions similarly to tweezers, in that it includes first and second levers 106a and 106b that are fixed together at a fulcrum 105 at the proximal end 102 of device 100 to permit the distal end clamping portion 104 to engage and hold an object when pinched together, such as a patient's eyelid tissue. There is no limitation to the size and shape of each of first and second levers 106a and 106b, or to fulcrum 105, other than it should be sized and shaped to be comfortably operated in a single hand of the practitioner.

At the distal end clamping portion 104 of device 100 are the clamping bars 109a and 109b, extending from levers 106a and 106b, respectively. Clamping bars 109a and 109b run parallel to each other and have opposing inner surfaces 110a and 110b that face each other. Inner surfaces 110a and 110b may be smooth or textured (e.g. grooves, ridges, indents, detents, recesses, protrusions, serrations, or any other desired structures).

Device 100 further includes a junction region 108 between each lever 106a or 106b and its respective clamping bar 109a or 109b. Junction region 108 sets the angle 112 at which levers 106a and 106b extend away from clamping bars 109a and 109b. In some embodiments, for example when clamping bars 109a and 109b are horizontal, levers 106a and 106b may extend away from the distal end clamping portion 104 at an angle 112 of between 0-90°, 0-180°, 0-270°, or 0-360°. In some embodiments, angle 112 is between 5-285°, between 10-280°, between 15-275°, between 20-270°, between 25-265°, between 30-260°, between 35-255°, between 40-250°, between 45-240°, between 50-235°, between 55-230°, between 60-225°, between 65-220°, between 70-215°, between 75-210°, between 80-205°, between 85-200°, between 90-195°, between 90-185°, between 90-175°, between 90-165°, between 90-155°, between 90-135°or between 90-125°. In some embodiments, angle 112 is about 5°, about 10°, about 15°, about 20°, about 25°, about 30°, about 35°, about 40°, about 45°, about 50°, about 55°, about 60°, about 65°, about 70°, about 75°, about 80°, or about 85°. In some embodiments, junction region 108 keeps angle 122 fixed. In some embodiments, junction region 108 may be adjustable, such that angle 112 is adjustable between 0-90°, 0-180°, 0-270°, or 0-360°. In some embodiments, junction 108 may be adjustable to 2, 3, 4, 5, 6, 7, 8, 9, 10, 11, 12, 13, 14, 15 lockable angular settings. Angle 112 may be also be described herein as having first and second angles for junction region 108, wherein the first angle describes the junction region 108 of lever arms 106a and clamping bars 109a, and the second angle describes the junction region 108 of second lever 106a and clamping bar 109b.

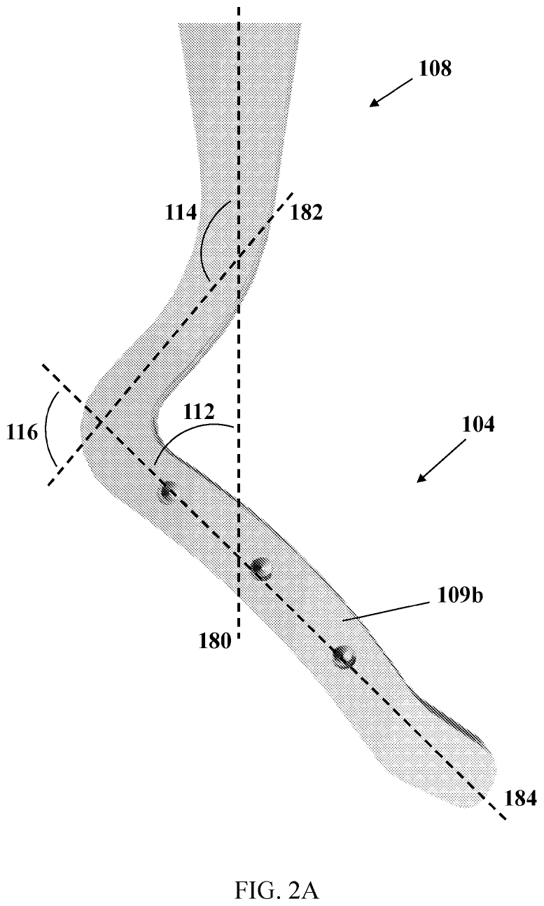

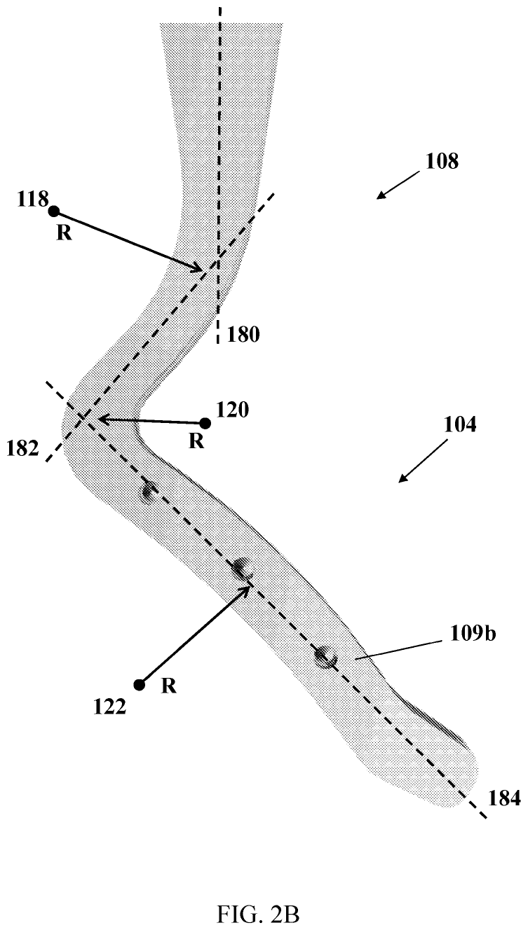

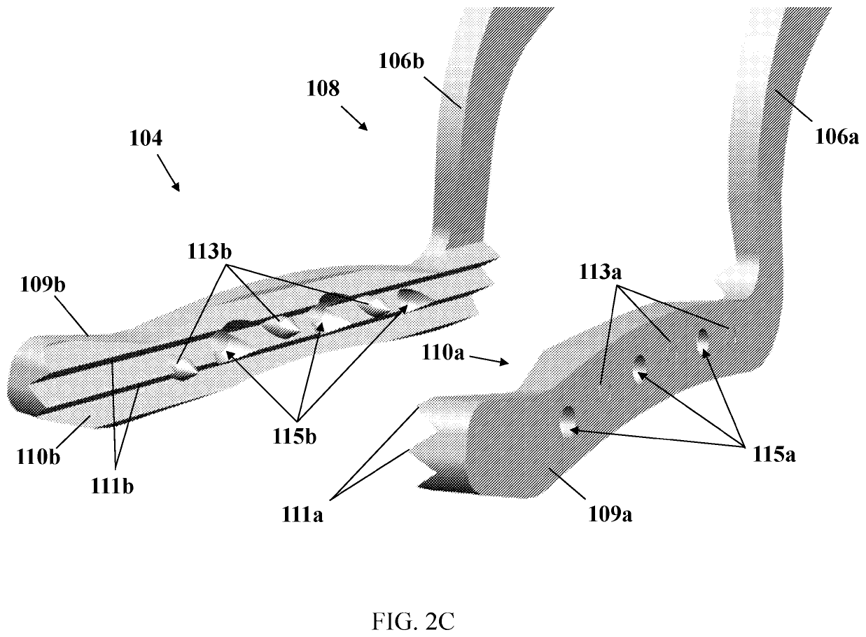

Referring now to FIG. 2A, an exemplary junction region 108 and distal end clamping portion 104 is shown. In some embodiments, junction region 108 is formed at the junction between first and second lever arms 106a and 106b and first and second clamping bars 109a and 109b, respectively. In some embodiments, junction region 108 extends from first and second lever arms 106a and 106b along a first axis 180, and redirects at a primary angle 114 to a second axis 182, and then redirects along a third axis 184 at a secondary angle 116 to form first and second clamping bars 109a and 109b. It should be appreciated herein that setting primary angle 114 and secondary angle 116 also sets angle 112 as described above. In some embodiments, primary angle 114 is between 0-180°. In some embodiments, primary angle 114 is between 105-180°, between 5-175°, between 10-170°, between 45-165°, between 55-160°, between 65-155°, between 75-150°, between 85-145°, between 95-140°, between 105-145°, between 115-140°, between 125-145°, or between 130 and 140°. In some embodiments, primary angle 114 is about 5°, about 10°, about 15°, about 20°, about 25°, about 30°, about 35°, about 40°, about 45°, about 50°, about 55°, about 60°, about 65°, about 70°, about 75°, about 80°, about 85°, about 90°, about 95°, about 100°, about 105°, about 110°, about 115°, about 120°, about 125°, about 130°, about 135°, about 140°, about 145°, about 150°, about 155°, about 160°, about 165°, about 170°, or about 175°.

In some embodiments, secondary angle 116 is between 60-165°, between 5-175°, between 10-170°, between 15-165°, between 20-160°, between 25-155°, between 30-150°, between 35-145°, between 40-140°, between 45-135°, or between 50-130°. In some embodiments, secondary angle 116 is about 5°, about 10°, about 15°, about 20°, about 25°, about 30°, about 35°, about 40°, about 45°, about 50°, about 55°, about 60°, about 65°, about 70°, about 75°, about 80°, about 85°, about 90°, about 95°, about 100°, about 105°, about 110°, about 115°, about 120°, about 125°, about 130°, about 135°, about 140°, about 145°, about 150°, about 155°, about 160°, about 165°, about 170°, or about 175°.

In some embodiments, device 100 is shown or described as having lever arms 106a and 106b extending upward at an angle 112 from distal end clamping portion 104 and/or junction region 108. However, in some embodiments lever arms 106a and 106b may extend downward at the equivalent of angle 112 from distal end clamping portion 104 and/or junction region 108 such that the practitioner is holding the body 101 to the side of the patient's face instead of above.

Referring in detail to FIG. 2B, in some embodiments, junction region 108 and/or distal end clamping portion 104 may comprise one or more curved portions having one or more radii. In some embodiments, junction region 108 extends along a first axis 180 and redirects with a first curved portion having a first radius 118 to a second axis 182, and then redirects with a second curved portion having a second radius 120 to a third axis 184. In some embodiments, clamping bars 109a and 109b each comprise at least one curved portion or region having one or more radii. For example, in some embodiments, clamping bars 109a and 109b comprise a third curved portion having a third radius 122. First radius 118, second radius 120, and/or third radius 122 may also be described as having an inner and outer radius, that are the same, similar, or different to one another. First radius 118, second radius 120, and/or third radius 122 may have any appropriate dimension or dimensions. In some embodiments, first radius 118, second radius 120, and/or third radius 122 have a radius ranging between 0.1 cm and 10 cm. For example, in some embodiments first radius 118 is about 1 cm, second radius 120 is about 0.5 cm, and third radius 122 is about 2.5 cm. In some embodiments, third radius 122 is larger than first radius 118, which is larger than second radius 120.

In some embodiments, clamping bars 109a and 109b are sized and shaped to follow and fit the size and shape of an eyelid. In some embodiments, clamping bars 109a and 109b may be sized and shape to fit an object or subject, or area of interest on a subject, such as the eyelids of a child, or an adult, or an animal.

Referring now to FIG. 2C, shown is an enlarged perspective view of an exemplary distal end clamping portion 104. Clamping bars 109a and 109b and/or inner surfaces 110a and 110b may comprise one or more features to facilitate precise and reliable gripping of an object. In some embodiments, inner surfaces 110a and 110b comprise one or more ridges 111a and 111b extending along at least a portion of each inner surface. In some embodiments, one or more ridges 111a and 111b are staggered such that each set of ridges at least partially enters into the area formed between the opposing set of ridges when clamping bars 109a and 109b are closed together. In some embodiments, clamping bars 109a and 109b each comprise one or more pins 113a and 113b, and one or more openings 115a and 115b positioned opposing each pin, configured such that each pin may at least partially enter the opposing opening when clamping bars 109a and 109b are closed together. In some embodiments, one or more pins 113a and 113b each comprise a conical shape, or each terminate at a point.

One or more ridges 111a and 111b may have any suitable size or shape in order to facilitate grip and/or produce friction when clamping bars 109a and 109b are gripping an object. In some embodiments, one or more ridges 111a and 111b have a height ranging between 0.01 cm and 0.5 cm, and a peak-to-peak distance ranging between 0.01 cm and 0.5 cm. In some embodiments, one or more ridges 111a and 111b comprise 1, 2, 3, 4, 5, 6, 7, 8, 9, or 10 ridges. Similarly, pins 113a and 113b may have any suitable size or shape to firmly hold an object between clamping bars 109a and 109b. In some embodiments, one or more pins 113a and 113b have a height, or extend out from inner surfaces 110a and 110b a distance ranging between 0.01 cm and 1 cm. In some embodiments, one or more openings 115a and 115b have a diameter or width ranging between 0.01 cm and 1 cm. In some embodiments, one or more openings 115a and 115b comprise chamfered edges or rims.

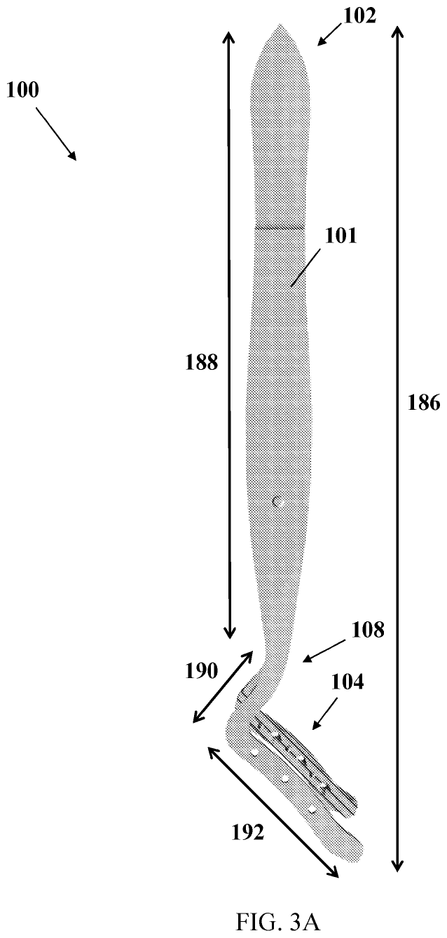

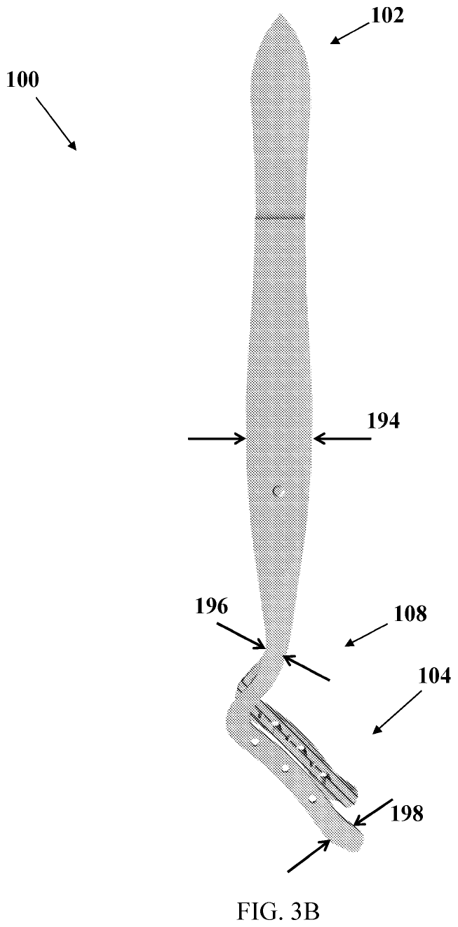

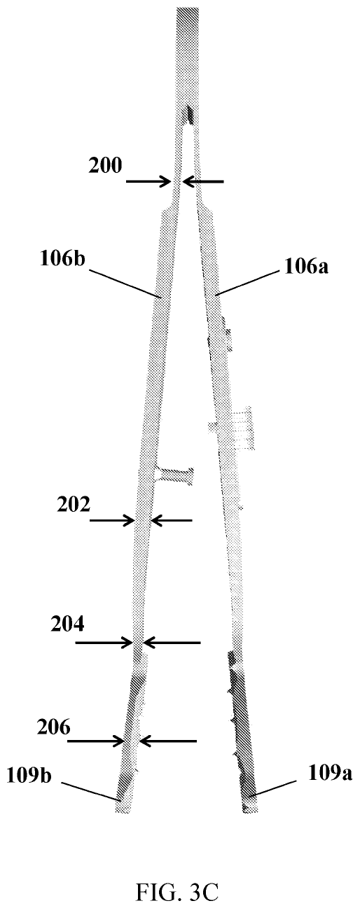

Referring now in detail to FIG. 3A, shown is a side view of an exemplary ptosis clamp device 100 with dimension lines. In some embodiments, body 101 has a length 186 ranging between 1 cm and 26 cm, between 2 cm and 24 cm, between 4 cm and 22 cm, between 6 cm and 20 cm, between 8 cm and 18 cm, or between 10 cm and 16 cm. In some embodiments, body 101 has a length 186 of about 2 cm, about 4 cm, about 6 cm, about 8 cm, about 10 cm, about 12 cm, about 13 cm, about 14 cm, about 15 cm, about 16 cm, about 17 cm, about 18 cm, about 19 cm, about 20 cm, about 21 cm, about 22 cm, about 23 cm, or about 24 cm.

In some embodiments, first and second levers 106a and 106b have a length 188 ranging between 1 cm and 24 cm, between 2 cm and 22 cm, between 4 cm and 20 cm, between 6 cm and 18 cm, between 8 cm and 16 cm, or between 10 cm and 14 cm. In some embodiments, first and second levers 106a and 106b have a length 188 of about 2 cm, about 4 cm, about 6 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, or about 22 cm.

In some embodiments, at least a portion of junction region 108 has a length 190 ranging between 0.1 cm and 5 cm, between 0.5 cm and 4.5 cm, between 1 cm and 4 cm, between 1.5 cm and 3.5 cm, or between 2 cm and 3 cm. In some embodiments, at least a portion of junction region 108 has a length 190 of about 0.1 cm, about 0.25 cm, about 0.5 cm, about 0.75 cm, about 1 cm, about 1.1 cm, about 1.2 cm, about 1.3 cm, about 1.4 cm, about 1.5 cm, about 1.6 cm, about 1.7 cm, about 1.8 cm, about 1.9 cm, about 2 cm, about 2.1 cm, about 2.2 cm, about 2.3 cm, about 2.4 cm, about 2.5 cm, about 2.75 cm, about 3 cm, about 3.25 cm, or about 3.5 cm.

In some embodiments, distal end clamping portion 104 and/or clamping bars 109a and 109b have a length 192 ranging between 0.1 cm and 6 cm, between 0.5 cm and 5.5 cm, between 1 cm and 5 cm, between 1.5 cm and 4.5 cm, between 2 cm and 4 cm, between 2.5 and 3.5 cm, or between 2.75 cm and 3.25 cm. In some embodiments, distal end clamping portion 104 and/or clamping bars 109a and 109b have a length 192 about 1 cm, about 1.1 cm, about 1.2 cm, about 1.3 cm, about 1.4 cm, about 1.5 cm, about 1.6 cm, about 1.7 cm, about 1.8 cm, about 1.9 cm, about 2 cm, about 2.1 cm, about 2.2 cm, about 2.3 cm, about 2.4 cm, about 2.5 cm, about 2.6 cm, about 2.7 cm, about 2.8 cm, about 2.9 cm, about 3 cm, about 3.25 cm, or about 3.5 cm.

Referring now to FIG. 3B, shown is a side view of an exemplary ptosis clamp device 100 with dimension lines. In some embodiments, body 101 and/or levers 106a and 106b have a width 194 ranging between 0.1 cm and 3 cm, between 0.2 cm and 2.9 cm, between 0.2 cm and 2.8 cm, between 0.3 cm and 2.7 cm, between 0.4 cm and 2.6 cm, between 0.5 cm and 2.5 cm, between 0.6 cm and 2.4 cm, between 0.7 cm and 2.3 cm, between 0.8 cm and 2.2 cm, between 0.9 cm and 2.1 cm, between 1 cm and 2 cm, or between 1.1 cm and 1.9 cm. In some embodiments, body 101 and/or levers 106a and 106b have a width 194 of about 0.25 cm, about 0.5 cm, about 0.75 cm, about 0.8 cm, about 0.9 cm, about 1 cm, about 1.1 cm, about 1.2 cm, about 1.3 cm, about 1.4 cm, about 1.5 cm, about 1.6 cm, about 1.7 cm, about 1.8 cm, about 1.9 cm, about 2 cm, about 2.1 cm, about 2.2 cm, about 2.3 cm, about 2.4 cm, or about 2.5 cm.

In some embodiments, junction region 108 has a width 196 ranging between 0.1 cm and 2 cm, between 0.15 cm and 1.9 cm, between 0.2 cm and 1.8 cm, between 0.2 cm and 1.7 cm, between 0.25 cm and 1.6 cm, between 0.3 cm and 1.5 cm, between 0.35 cm and 1.45 cm, between 0.4 cm and 1.4 cm, between 0.45 cm and 1.35 cm, or between 0.5 cm and 1.3 cm. In some embodiments, junction region 108 has a width 196 of about 0.1 cm, about 0.15 cm, about 0.2 cm, about 0.25 cm, about 0.3 cm, about 0.35 cm, about 0.4 cm, about 0.45 cm, about 0.5 cm, about 0.55 cm, about 0.6 cm, about 0.65 cm, about 0.7 cm, about 0.75 cm, about 0.8 cm, about 0.85 cm, about 0.9 cm, about 0.95 cm, or about 1 cm.

In some embodiments, distal end clamping portion 104 and/or clamping bars 109a and 109b have a width 198 ranging between 0.1 cm and 2 cm, between 0.15 cm and 1.9 cm, between 0.2 cm and 1.8 cm, between 0.2 cm and 1.7 cm, between 0.25 cm and 1.6 cm, between 0.3 cm and 1.5 cm, between 0.35 cm and 1.45 cm, between 0.4 cm and 1.4 cm, between 0.45 cm and 1.35 cm, or between 0.5 cm and 1.3 cm. In some embodiments, distal end clamping portion 104 and/or clamping bars 109a and 109b have a width 198 of about 0.1 cm, about 0.15 cm, about 0.2 cm, about 0.25 cm, about 0.3 cm, about 0.35 cm, about 0.4 cm, about 0.45 cm, about 0.5 cm, about 0.55 cm, about 0.6 cm, about 0.65 cm, about 0.7 cm, about 0.75 cm, about 0.8 cm, about 0.85 cm, about 0.9 cm, about 0.95 cm, or about 1 cm.

Referring now to FIG. 3C, shown is a front view of an exemplary ptosis clamp device 100 with dimension lines. In some embodiments, body 101 and/or first and second levers 106a and 106b have a first or proximal thickness 200 ranging between 0.05 cm and 1 cm, between 0.1 cm and 0.1 cm, between 0.15 cm and 0.95 cm, between 0.2 cm and 0.9 cm, between 0.25 cm and 0.85 cm, between 0.3 cm and 0.8 cm, between 0.35 cm and 0.75 cm, between 0.4 cm and 0.7 cm, between 0.45 cm and 0.65 cm, or between 0.5 cm and 0.6 cm. In some embodiments, first and second levers 106a and 106b have a first or proximal thickness 200 of about 0.1 cm, about 0.15 cm, about 0.2 cm, about 0.25 cm, about 0.3 cm, about 0.4 cm, about 0.5 cm, about 0.6 cm, about 0.7 cm, about 0.8 cm, about 0.9 cm or about 1 cm.

In some embodiments, body 101 and/or levers 106a and 106b have a second or distal thickness 202 ranging between 0.05 cm and 1 cm, between 0.1 cm and 1 cm, between 0.15 cm and 0.95 cm, between 0.2 cm and 0.9 cm, between 0.25 cm and 0.85 cm, between 0.3 cm and 0.8 cm, between 0.35 cm and 0.75 cm, between 0.4 cm and 0.7 cm, between 0.45 cm and 0.65 cm, or between 0.5 cm and 0.6 cm. In some embodiments, body 101 and/or levers 106a and 106b have a second or distal thickness 202 about 0.1 cm, about 0.15 cm, about 0.2 cm, about 0.25 cm, about 0.3 cm, about 0.4 cm, about 0.5 cm, about 0.6 cm, about 0.7 cm, about 0.8 cm, about 0.9 cm or about 1 cm.

In some embodiments, junction region 108 has a thickness 204 ranging between 0.05 cm and 1 cm, between 0.1 cm and 0.1 cm, between 0.15 cm and 0.95 cm, between 0.2 cm and 0.9 cm, between 0.25 cm and 0.85 cm, between 0.3 cm and 0.8 cm, between 0.35 cm and 0.75 cm, between 0.4 cm and 0.7 cm, between 0.45 cm and 0.65 cm, or between 0.5 cm and 0.6 cm. In some embodiments, junction region 108 has a thickness 204 of about 0.1 cm, about 0.15 cm, about 0.2 cm, about 0.25 cm, about 0.3 cm, about 0.4 cm, about 0.5 cm, about 0.6 cm, about 0.7 cm, about 0.8 cm, about 0.9 cm or about 1 cm.

In some embodiments, distal end clamping portion 104 and/or clamping bars 109a and 109b have a thickness 206 ranging between 0.05 cm and 1 cm, between 0.1 cm and 1 cm, between 0.15 cm and 0.95 cm, between 0.2 cm and 0.9 cm, between 0.25 cm and 0.85 cm, between 0.3 cm and 0.8 cm, between 0.35 cm and 0.75 cm, between 0.4 cm and 0.7 cm, between 0.45 cm and 0.65 cm, or between 0.5 cm and 0.6 cm. In some embodiments, distal end clamping portion 104 and/or clamping bars 109a and 109b have a thickness 206 of about 0.1 cm, about 0.15 cm, about 0.2 cm, about 0.25 cm, about 0.3 cm, about 0.4 cm, about 0.5 cm, about 0.6 cm, about 0.7 cm, about 0.8 cm, about 0.9 cm or about 1 cm.

It should be appreciated that regions or portions of device 100 (e.g., body 101, levers 106a, 106b, junction region 108, distal end clamping portion 104, clamping bars 109a, 109b) may comprise one length, height or width, or may comprise more than one length, height or width. For example, in some embodiments, regions or portions of device 100 comprise curved regions with first and second lengths, heights or widths selected from any dimensions contemplated herein. Further, device 100 may be formed from any suitable material, such as rigid and non-porous materials, metals, metal alloys, plastics, polymers, 3D printed materials, combinations thereof, and/or any sterilizable materials that may be used for tweezers or forceps that would be known and applied by one of ordinary level of skill in the art.

Device 100 may further comprise a locking mechanism 107 to lock and unlock device 100 (i.e., first and second levers 106a and 106b to each other). Locking mechanism 107 may comprise a single component, or plurality of components configured to lock the levers together once compressed. Once the levers are compressed and locking mechanism 107 is engaged, the practitioner may release compression and the levers will stay compressed and locked together, thus gripping the object (e.g., the eyelid, eye tissue). In some embodiments, locking mechanism 107 is adjustable, and/or can be configured in various positions to provide a variable compression force on the gripped object.

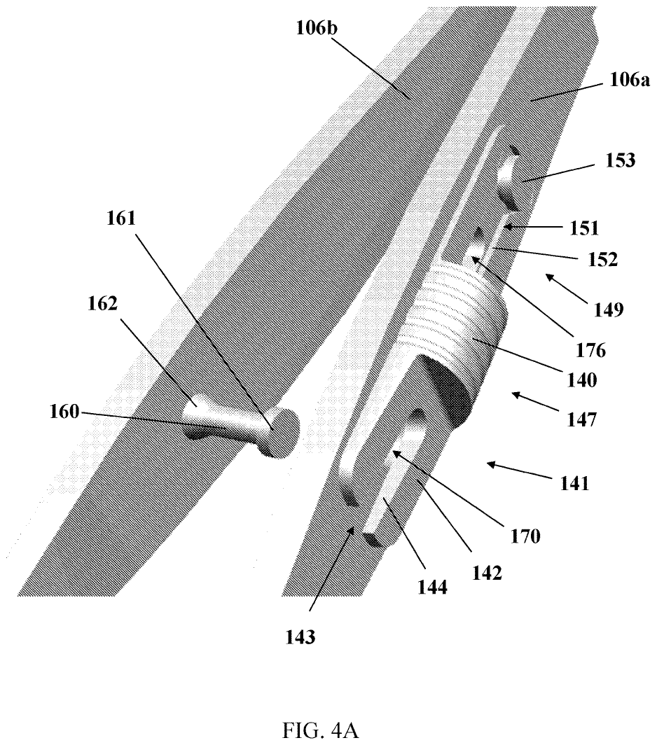

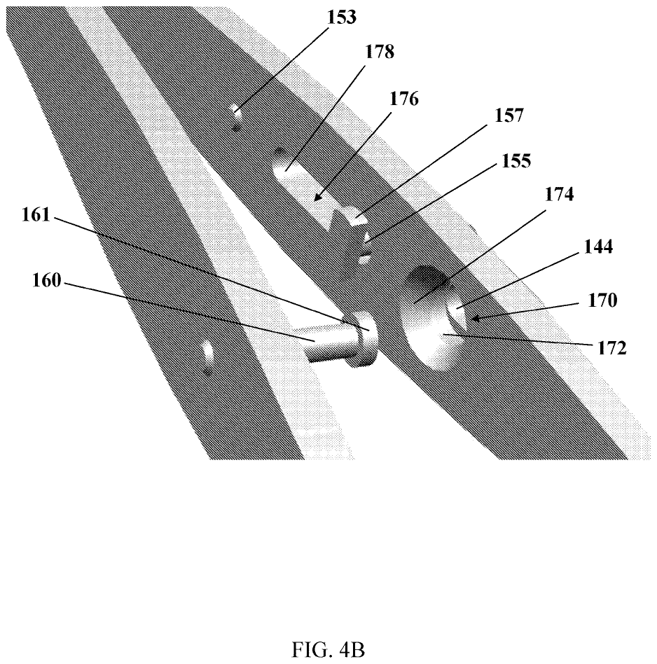

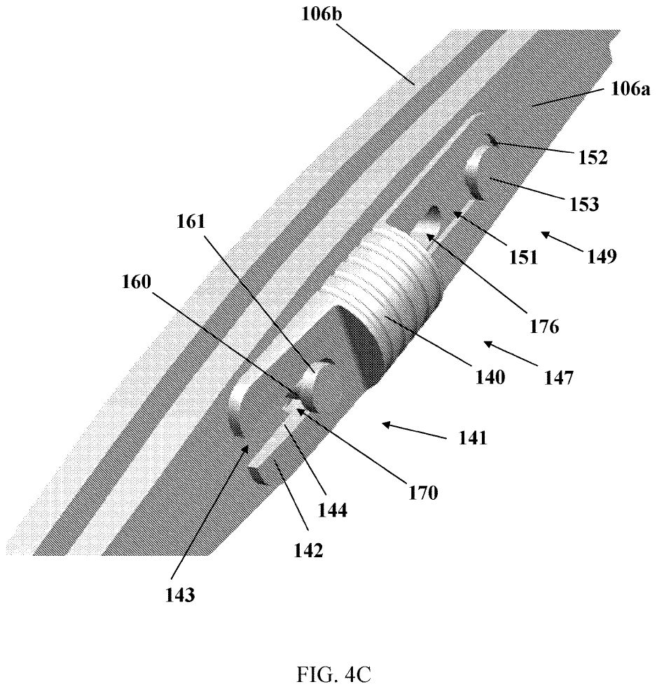

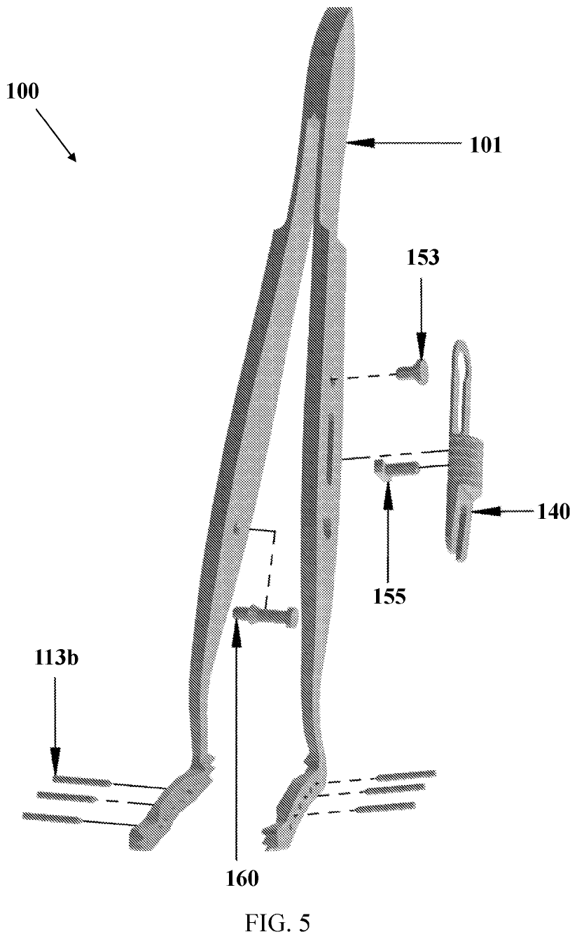

Referring now to FIGS. 4A through 4C, an exemplary locking mechanism 107 for device 100 is depicted. In some embodiments, locking mechanism 107 comprises a catch 140 slidably attached to first lever 106a, and a receiver 160 with a distal flange 161 fixedly attached to second lever 106b. In order to lock the locking mechanism 107, a portion of receiver 160 passes through a first opening 170 in first lever 106a to engage catch 140 and lock and unlock device 100 (a locked device 100 is shown in FIGS. 1C & 4C). In some embodiments, catch 140 comprises a catching portion 141, a gripping portion 147, and a retaining portion 149. In some embodiments, catching portion 141 comprises a ramp 142 having a catch opening 143 forming sidewalls 144. When catch 140 is pushed in a distal direction to lock device 100, catch opening 143 and sidewalls 144 are sized and shaped to receive and releasably hold at least a portion of receiver 160 (e.g., distal flange 161). In some embodiments, distal flange 161 is sized and shaped to be retained and releasably held within catch opening 143 by ramp 142 and sidewalls 144.

In some embodiments, receiver 160 further comprises a proximal chamfer 162 to further align and center receiver 160 at least partially within first opening 170 when device 100 is closed and/or locked. In some embodiments, ramp 142 and/or sidewalls 144 have a lower distal height and higher proximal height to provide an adjustable range for locking and/or setting the clamping pressure of device 100. In some embodiments, catch opening 143 and/or sidewalls 144 have a wider distal width and narrower proximal width configured to hold receiver 160 when catch 140 is pushed in a distal direction.

In some embodiments, retaining portion 149 comprises an opening 151 forming sidewalls 152 configured to slide along, and be at least partially retained by a retaining pin 153 that is fixedly attached to first lever arm 106a. In some embodiments, catch 140 comprises a sliding post 155 extending downward from catch 140 at least partially through a second opening 176 in first lever 106a. In some embodiments, sliding post 155 comprises a slide 157 sized and shaped to slide within and be at least partially retained by second opening 176 in first lever 106a.

In some embodiments, first opening 170 forms a sidewall 172 comprising a chamfered portion 174. Sidewall 172 and chamfered portion 174 can be any suitable size and shape to direct and align flange region 161 and/or receiver 160 to at least partially pass through first opening 170. In some embodiments, first opening 170 has a diameter ranging between 0.1 cm and 2 cm. In some embodiments, second opening 176 has a height or first diameter ranging between 0.1 cm and 2 cm, and a width or second diameter ranging between 0.1 cm and 2 cm. In some embodiments, first opening 170 is a round shape and second opening 176 is a slotted or oval shape.

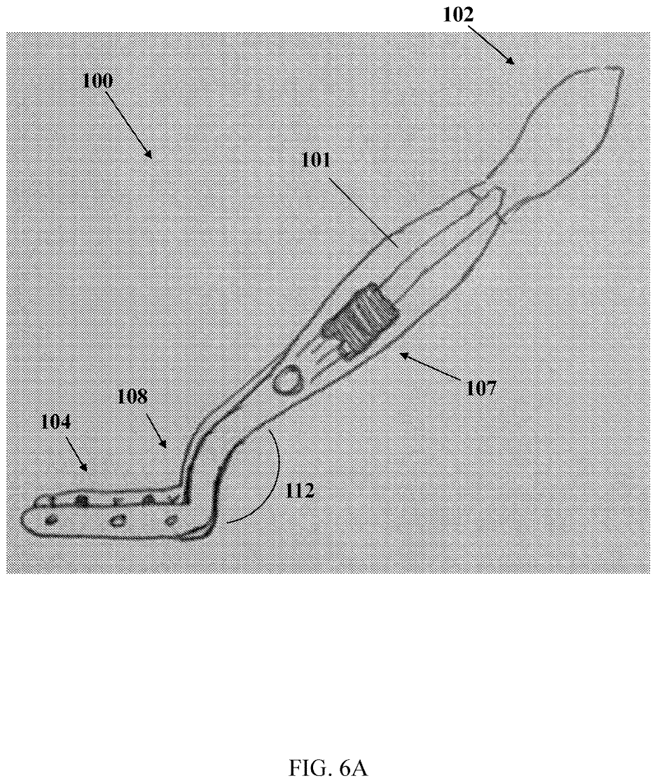

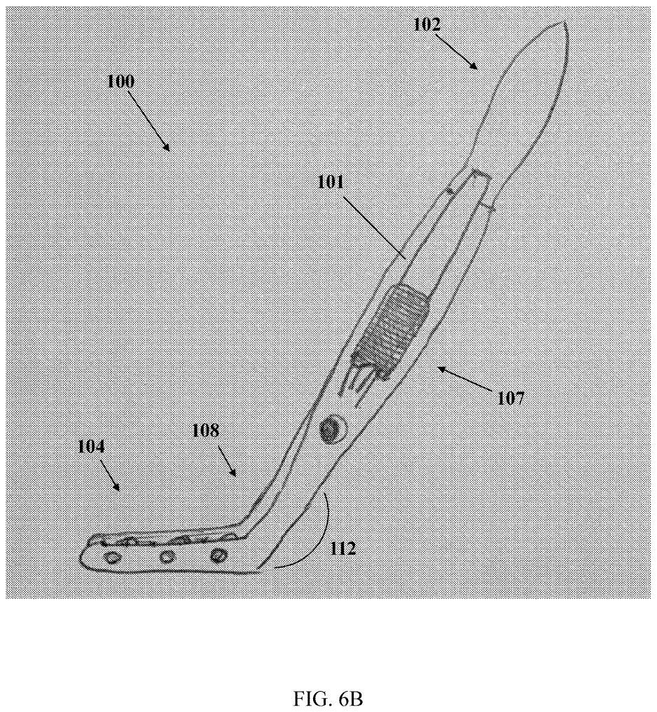

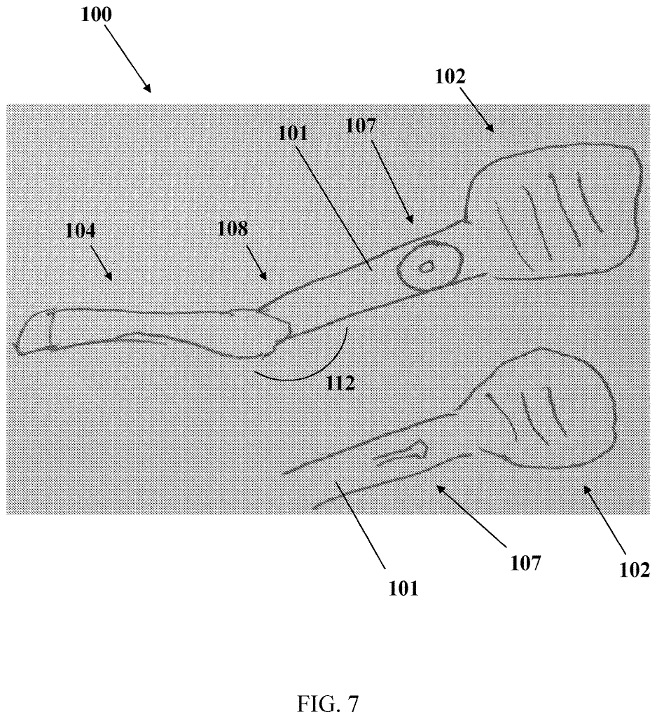

FIGS. 6A, 6B & 7 show alternative embodiments of exemplary ptosis clamp devices according to aspects of the present invention.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

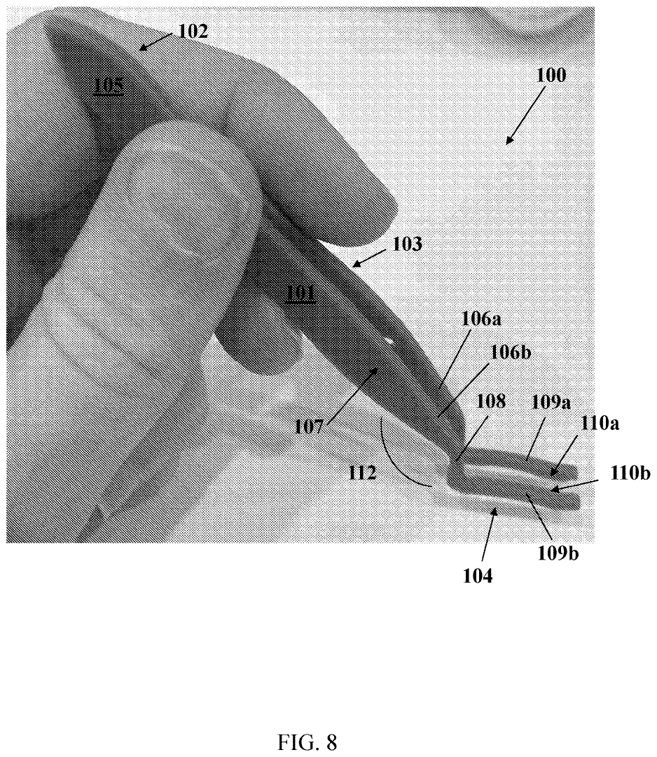

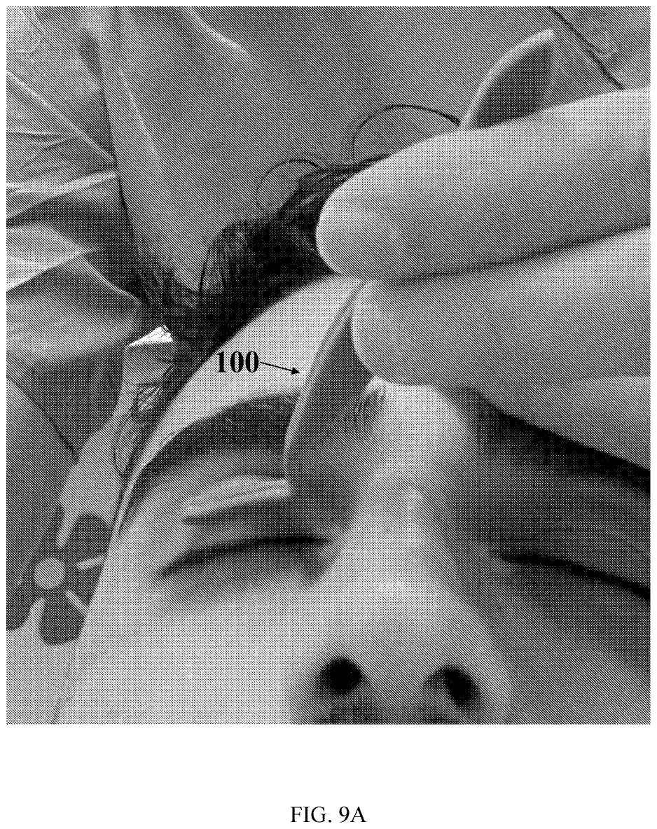

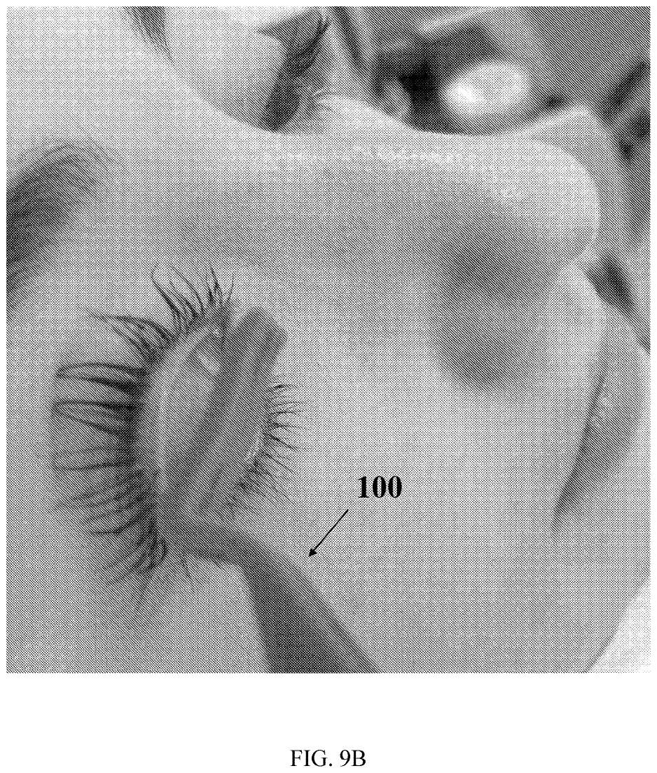

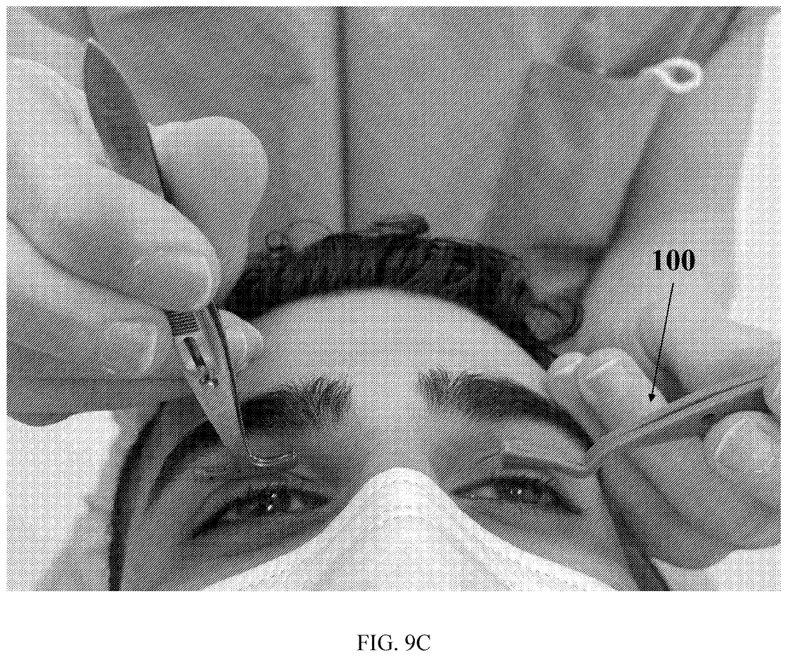

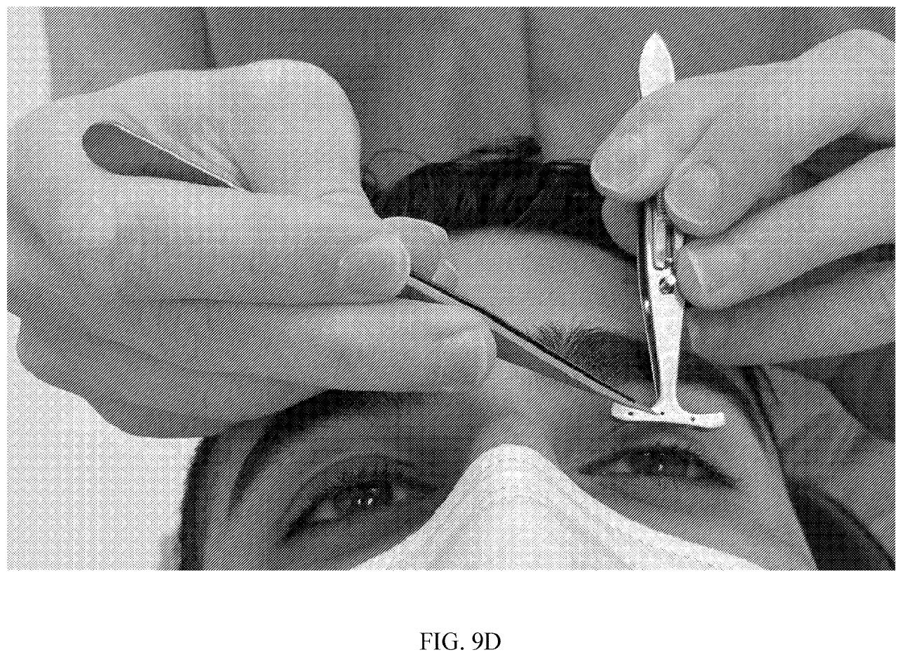

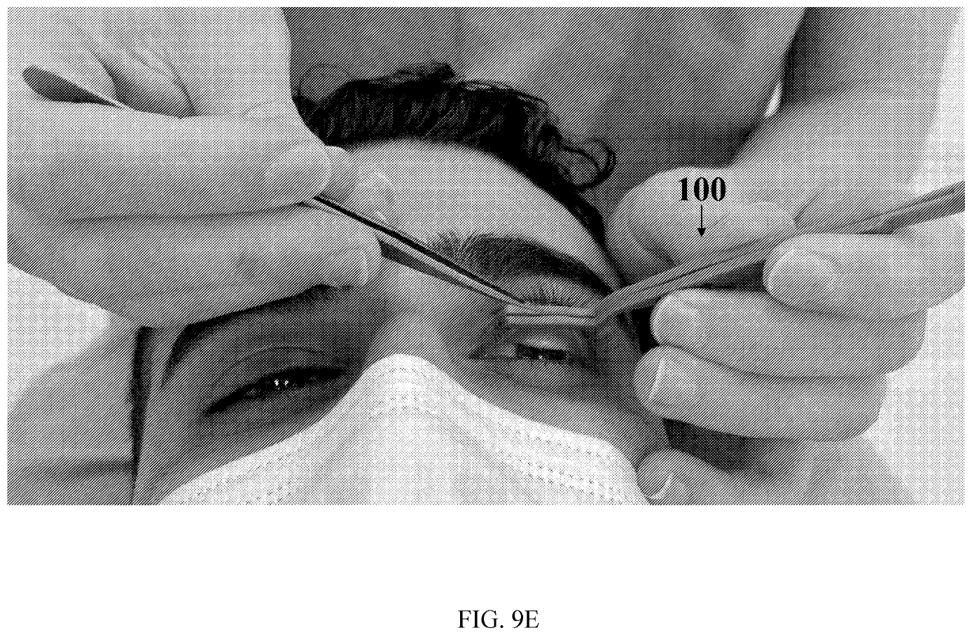

A prototype of an exemplary ptosis clamp device (e.g., a device 100) was constructed. FIG. 8 is an image of a prototype of an exemplary ptosis clamp device. FIG. 9A illustrates the exemplary ptosis clamping device of FIG. 8, with the clamping portion positioned at the patient's upper eyelid and the grasping handle extending across the midline over the bridge of the patient's nose. As seen, the practitioner's hand can hold and operate the ptosis clamp without obstructing their view of the patient's eyelid being clamped. FIG. 9A is illustrative in a similar manner as FIG. 9B, except the levers are angled such that they extend away from the midline of the patient's head, again providing superior visualization of the patient's eyelid and easy access to the eyelid tissues.

FIG. 9C is illustrative of the differences in the practitioner's hand positioning when operating the ptosis clamp device described herein verses a standard Putterman clamp. The Putterman clamp is positioned on the upper eyelid of the patient's right eye, while the ptosis clamp device of FIG. 8 is positioned on the upper eyelid of the patient's left eye. As is readily apparent, the practitioner's hand holding the Putterman clamp is within the practitioner's line of sight, thereby partially obstructing the practitioner's visualization of the patient's eyelid being clamped. In contrast, the ptosis clamp device FIG. 8 allows the practitioner's hand to hold and operate the ptosis clamp without obstructing their view of the patient's eyelid being clamped.

FIG. 9D further illustrates the usage of the Putterman clamp in conjunction with a second instrument. The practitioner must move the clamp forward and backward in order to gain a proper visualization of the eyelid tissue to manipulate with the second instrument. This is in contrast to use of the ptosis clamp device of FIG. 8 as shown in FIG. 9E. As shown, the device of FIG. 8 can engage the patient's eyelid with the levers extending away from the midline of the patent's head, thereby permitting full visualization by the practitioner and improved access to the eyelid tissues with the second instrument.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A ptosis clamp device, comprising:
a first lever having a proximal end and a distal end;
a second lever having a proximal end and a distal end, wherein the first and second levers are fixed at their proximal ends to form a fulcrum;
a first clamping bar;
a first junction connecting the distal end of the first lever to the first clamping bar;
a second clamping bar; and
a second junction connecting the distal end of the second lever to the second clamping bar,
wherein a first angle of the first lever relative to the first clamping bar is between 40-250°;
wherein a second angle of the second lever relative to the second clamping bar is between 40-250°; and
wherein the first and second angles are equal.

2. The device of claim 1, wherein the first and second angles are between 50-200°.

3. The device of claim 1, wherein the first and second angles are between 60-180°.

4. The device of claim 1, wherein the first and second angles are between 90-150°.

5. The device of claim 1, wherein the first and second angles are adjustable within the first and second junctions, respectively.

6. The device of claim 1, wherein the first and second clamping bars each comprise at least one curved portion.

7. The device of claim 1, wherein the first and second junctions each comprise at least one curved portion.

8. The device of claim 1, wherein the first and second clamping bars each have an opposing, inner facing surface that is smooth.

9. The device of claim 1, wherein the first and second clamping bars each have an opposing, inner facing surface that is textured.

10. The device of claim 9, wherein each texture surface comprises any of indents, grooves, ridges, detents, or serrations.

11. The device of claim 1, wherein the first and second clamping bars each have an opposing, inner facing surface with one or more pins extending outward from each surface.

12. The device of claim 11, wherein the first and second bars each have one or more openings opposing each pin and at least partially passing through the respective bar.

13. The device of claim 12, wherein each opening passes fully through the respective bar.

14. The device of claim 13, wherein each opening comprises a chamfered portion.

15. The device of claim 1, further comprising a locking mechanism attached to the first and second levers configured to releasably lock the levers together.

16. The device of claim 15, wherein the locking mechanism comprises a receiver attached to the first lever, an opening in the second lever, and a catch slidably attached to the second lever over the opening, wherein the receiver at least partially passes through the opening and engages the catch.

17. The device of claim 16, wherein the opening in the second lever comprises a chamfered portion configured to align the receiver to the center of the opening.

18. The device of claim 17, wherein the catch comprises a ramped portion having a ramp with a catch opening configured to releasably engage the receiver in one or more positions on the ramp.

19. The device of claim 18, wherein the receiver comprises a distal flanged portion configured to engage with the catch opening and ramp.

20. The device of claim 19, wherein the catch opening has sidewalls forming a proximal narrow portion and a distal wider portion.
